(19) United States
(12) Patent Application Publication
(10) Pub. No.: US 2017/0027744 A1
(43) Pub. Date: Feb. 2, 2017

(54) Cooling blanket for humans and animals

(71) Applicant: Bonnie Iris Floyd, Baton Rouge, LA (US)

(72) Inventor: Bonnie Iris Floyd, Baton Rouge, LA (US)

(21) Appl. No.: 15/004,938

(22) Filed: Jan. 23, 2016

Related U.S. Application Data
(60) Provisional application No. 62/107,139, filed on Jan. 23, 2015.

(51) Int. Cl. A61F 7/10

(52) U.S. Cl. CPC A61F 7/10; A61F 7/106; A61F 2007/108

(57) ABSTRACT

A cooling blanket, singularly or in plurality, which covers the core body of humans or animals to facilitate immediate, rapid cooling through sweat evaporation and radiant heat exchange by means of an integrated ventilation system and maximum body surface contact via an incorporated cooling medium contained within multiple, sealed compartments within the embodiment.

### CROSS-REFERENCE TO RELATED APPLICATION

This application claims the benefit of PPA Ser. No. 62/107,139, filed 2015 Jan. 23 by the present inventor, which is incorporated by reference.

Cold/Warm Blanket utilizing specialized fabrics and a pliable gel that does not freeze at low temperatures and can be heated to a wide range of warm temperatures for the immediate, affordable, on-site treatment of hyperthermia or hypothermia to quickly reduce hot body temperatures or gradually warm cold body temperatures, respectively, and can remain with the patient throughout transport without interfering with other treatments

### FIELD OF ART

Related to the field of medical equipment, especially to personal cooling blankets

### BACKGROUND

1) Every year, a number of athletes, migrant workers, construction workers, soldiers, and other persons in hot environments, and animals, suffer damage to the brain and/or other vital organs, or die, because they do not have access to a sufficient, immediate, on-site treatment to lower core body temperature in the event of a heat stroke. The time between identifying a potential heat illness, heat stroke problem and treatment is crucial to a successful outcome. Lowering the body temperature just 1-2 degrees makes the difference between heat exhaustion and heat stroke and could make the difference between permanent damage and little or no damage, or between life and death.

The current recommendation for the immediate, on-site treatment of overheated persons is to immerse the person in an ice bath. In lieu of an ice bath, a cold shower, or placing cold water soaked towels over as much of the body as possible is the recommended treatment (“Heat Stroke Treatment”, “How do you treat an individual with heat stroke?”, University of Connecticut, Korey Stringer Institute, http://ksi.uconn.edu/emergency-conditions/heat-illnesses/exertional-heat-stroke/heat-stroke-treatment/). Additionally, placing cold packs on the neck, armpits, and groin is also recommended (“Heatstroke: First aid”, Mayo Clinic Staff, Mar. 31, 2015, http://www.mayoclinic.org/first-aid/first-aidheatstroke/basics/ART-20056655,). The vast majority of schools and public sports programs, as well as businesses in agriculture, construction, military in the field, and many other hot work environments, do not have on-site facilities for ice baths or access to running cold water, large quantities of ice, or transport and storage options for either.

2) Studies now show that the immediate cooling of cardiac arrest victims could result in a reduction in damage to the heart and other organs (“Hypothermia and cardiac arrest: the promise of intra-arrest cooling”, Roger A. Band and Benjamin S. Abella, Apr. 22, 2008, Critical Care, article reprint online US National Library of Medicine, National Institutes of Health, http://www.ncbi.nlm.nih.gov/pmc/articles/PMC2447582/). Employers, coaches, event managers, and others, and non-transport emergency response teams have very few options for on-site cooling of individuals. For these responders, on-site treatments are usually limited to the use of individual cold packs or limited amounts of ice. Most emergency medical transport teams are limited to the administering of cold intravenous fluids and the application of small cold packs.

Cooling blankets that currently exist fall into the following categories: 1) cooling blankets large enough to cool the torso or entire body utilizing mechanical cooling systems requiring a power source and not portable enough to be used outside medical facilities, 2) cooling blankets requiring activation with water, or with a mixture of water, a thixotropic filler, and a water freezing point depressant, or containing pockets for cold packs of some form, 3) single use cooling blankets that contain reagents that mix within the compartments of the blanket itself for an endothermic reaction, and 4) multiple-use or single-use smaller cooling products containing a variety of gels, beads, oils, water based fillers, or a combination thereof, for cooling small areas or specific body parts.

None of these afore referenced blankets contain a ventilation system to allow the natural body cooling process of sweat evaporation. Without a ventilation system for removing warm air generated by body heat, warm air will be trapped in the voids between the body and the blanket seams, lessening the speed of core body cooling. The construction of current blankets large enough to rapidly cool a large adult make them either not portable enough, too expensive for general application, or both, and therefore, do not fill the need for an affordable, reusable cooling blanket for immediate, on-site use. While some cooling blankets can be rolled or folded to a smaller size, they do not fold compactly enough to make them easily transportable.

Thinly filled gel and wax blankets are not discussed because their cooling capacity is limited to small changes in normal body temperature and do not apply to the needs of a quick body cool down.

The cooling blanket, singularly or in plurality, solves the problem of the immediate, on-site treatment of heat-related illnesses, such as heat exhaustion and heat stroke, and in the case of deleterious health events, such as cardiac arrest. The cooling blanket allows for continued treatment during transport to a medical facility. The simplicity of the cooling blanket, its ease of use, and its use of readily available components in manufacturing, make it affordable to the vast majority of school sports programs, especially football programs where most athletic heat stroke deaths occur and in agriculture where most migrant worker deaths occur. The width of the seams housing the ventilation system allow the cooling blanket to be easily folded to a compact size for cooling in refrigerators, freezers, or with ice in ice chests, and transported to work sites in small ice chests or insulated bags, such as in construction where roofers and welders are especially susceptible to heat stroke, in military basic training, and in numerous other hot work environment fields where deaths continue to occur from heat stroke and cardiac arrest. Compactness for transport and storage is especially important in emergency response and transport vehicles where space is of utmost importance. The ventilation system in the cooling blanket, formed from a series of ventilation openings within the seams, allows the body to help cool itself with sweat evaporation and by preventing a layer of body-warmed air from being trapped in the blanket's seams. Additionally, radiant heat transfer takes place in the capillaries in the skin between the body's surface and the cold medium contained in the individualized cooling medium compartments within the blanket. The cooling medium stays pliable at temperatures below 32 F so that the cooling blanket conforms to the shape of the body to be cooled. Where there are no means for cooling the blanket for use, such as remote training areas, single use chemical reagents within the blanket may be utilized. The types of materials in the layers used to form the basis of the cooling blanket along with the bonding of its seams make the cooling blanket strong enough to support the body weight of humans or large animals without rupture. By placing one cooling blanket underneath the human or animal and draping a second cooling blanket over the top of the human or animal, the body's core, or the entire body, can be encompassed in immediate cold therapy. No special instructions are needed for using the blanket other than covering as much of the body's core as possible.

### SUMMARY

The cooling blanket is used on humans or animals for the immediate, on-site prevention or treatment of hyperthermia, such as heat exhaustion and heat stroke, and deleterious medical events, such as cardiac arrest, by wrapping the body's core in immediate cold therapy. Heat exchange takes place through the body's natural cooling process, i.e. evaporation of body sweat, via the ventilation system made up of a series of openings within the seams of the blanket, and through radiant heat transfer between the capillaries in the skin and the cold cooling medium in the blanket. The combination of these heat exchange processes quickly begins and continues lowering the core body temperature, preventing or limiting damage to the brain and/or other vital organs, and/or preventing death. The seams housing the ventilation system provide a flexibility that allows the blanket to be folded compactly for cooling, transport, and storage, especially important in emergency response and medical transport vehicles. The cooling medium can be a non-reagent gel that remains pliable below 32 F so that the blanket conforms to the body and which maintains its purpose and pliability through repeated uses. Mixing reagents may also be used as a cooling medium to produce special use blankets, i.e. blankets that may only be used one time or blankets needed in locations where no refrigeration system for pre-cooling the blankets is available.

### DESCRIPTION OF DRAWINGS

FIG. 1—A top view of the cooling blanket

FIG. 1A—A top view of a cooling blanket in a different configuration

FIG. 2—Close up of the ventilation system

FIG. 3—The construction layers of the cooling blanket

FIG. 3A—Construction layers made of one or more layers of material

FIG. 4—Cross section of the cooling blanket showing the individualized compartments and seams

### DETAILED DESCRIPTION

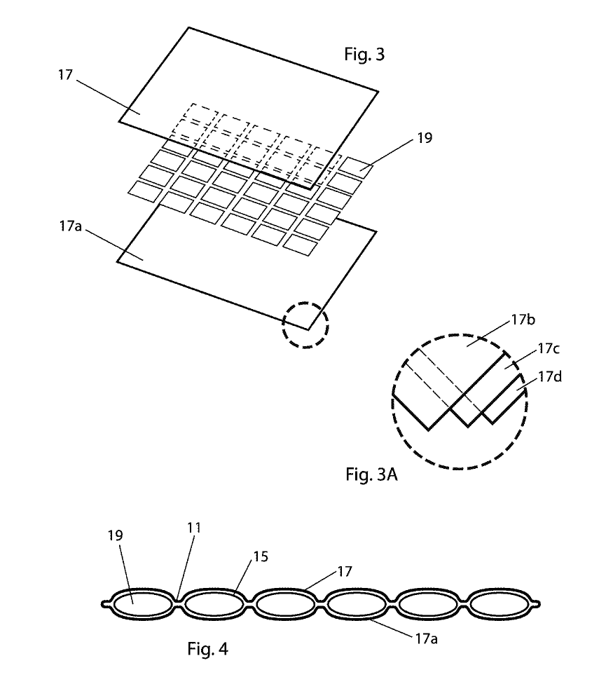

As illustrated in FIGS. 3 & 4, the cooling blanket is made up of a top layer of material 17, a layer of cooling medium 19, and a bottom layer of material 17.

As illustrated in FIG. 3A, the layers 17 and 17a can be any number of layers of materials 17b, 17c, and 17d.

As illustrated in FIGS. 3 & 4, the cooling medium 19 can be any type of cooling medium which remains pliable enough at temperatures below 32 F to conform to the core body of a human or animal.

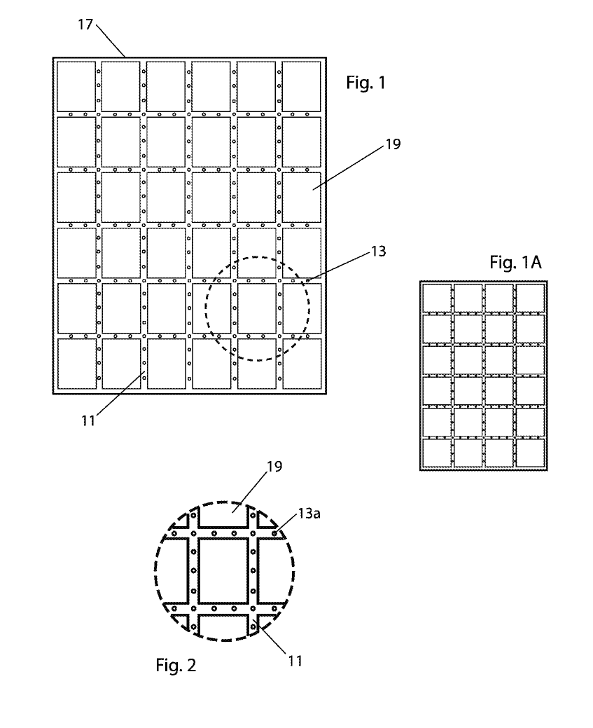

As illustrated in FIGS. 1, 2 & 4, the cooling blanket is constructed via a plurality of seams, welds, or bonds 11.

As illustrated in FIG. 4, the seams 11 within the body and around the edges of the cooling blanket form individualized compartments 15 for holding said cooling medium.

As illustrated in FIG. 2, the seams in the cooling blanket are in widths, which allow the integrity of the embodiment to be maintained, and in which a series of ventilations openings 13a of any size or shape are incorporated in the seams 11.

As illustrated in FIG. 1, the combination of the ventilation openings 13a in the seams 11 produce a ventilation system whereby air can pass through unhindered. The seams 11 become the bases for the easy folding of the embodiment to a compact size for cooling, transport, and/or storage.

As illustrated in FIGS. 1 & 1A, the embodiment can be made in any size suitable for cooling the body core, or entire body, of any human or animal.

The embodiment is cooled in a refrigerator, freezer, or in an ice chest with ice, dry ice, or other cooling capable medium. Cooling and storing the embodiment in an insulated container allows it to be transported to any location where immediate cooling of a person or animal may be needed. The embodiment may be left in any continuous cooling device for an indefinite amount of time or stored in an insulated container for use within a few hours or a few days, depending on the type of insulated container used.

While my description contains many specifications, these should not be construed as limitations on the scope, but rather as an exemplification of several embodiments. Many other variations are possible. For example, the series of said seams can take a configuration of curves instead of right angles, or the shape of the embodiment may change to allow for a continuous section to be drawn together around the head as well as to extend beneath the body with a separate embodiment draped over the body.

1. A cooling blanket, singularly or in plurality, that covers a human's or animal's core body, incorporating a non-mechanical ventilation system allowing for evaporation of sweat and body heat in conjunction with radiant heat transfer, whereby immediate, rapid cooling is facilitated in overheated humans or animals, or humans suffering a deleterious cardiac event such as cardiac arrest or other medical event, comprising
a. The embodiment of claim 1, wherein two layers of material large enough to cover the body core of an adult and made up of one or more sheets of various materials are connected at the edges and at intervals to form seams, bonds, or welds and individualized compartments in plurality
b. The embodiment of claim 1 and 1a, wherein said ventilation system consists of openings within said seams to facilitate the escape of body heat and sweat
c. The embodiment of claim 1, 1a, and 1b, wherein the series of said seams are made wide enough to support said openings without compromising the integrity of said compartments
d. The embodiment of claim 1 and 1a, wherein said compartments encapsulate a non-reagent cooling medium which remains pliable at temperatures below 32 F and through many re-cooling cycles
e. The embodiment of claim 1 and 1a, wherein the said cooling medium consists of reagents within said compartments that, when mixed within the compartments, produce an endothermic reaction and remain pliable
